(19) United States
(12) Patent Application Publication
(10) Pub. No.: US 2024/0298658 A1
(43) Pub. Date: Sep. 12, 2024

(54) FOOD FRESHNESS MAINTENANCE SYSTEM AND FRESHNESS MAINTENANCE AND TRANSFER DEVICE

(71) Applicant: AWEXOME RAY, INC., Anyang-si (KR)

(72) Inventors: Hong Sue CHOI, Anyang-si (KR); Hyungsub PARK, Anyang-si (KR); Ki Hoon YOON, Anyang-si (KR); Nam Kyu LEE, Anyang-si (KR); Se Hoon GIHM, Anyang-si (KR)

(21) Appl. No.: 18/664,843

(22) Filed: May 15, 2024

Related U.S. Application Data
(63) Continuation of application No. PCT/KR2021/016987, filed on Nov. 18, 2021.

(30) Foreign Application Priority Data
Nov. 18, 2021 (KR) 10-2021-0159029

(51) Int. Cl. A23B 7/152; B01D 53/32; B03C 3/32; B03C 3/47

(52) U.S. Cl. CPC A23B 7/152; B01D 53/323; B03C 3/32; B03C 3/47; B01D 2257/7022; B01D 2257/91; B01D 2259/4525; B01D 2259/4566; B03C 2201/30

(57) ABSTRACT

A food freshness maintenance system is proposed. The system may include a chamber configured to accommodate food and a freshness maintenance device installed in the chamber. The freshness maintenance device may include a main body including an inlet through which air flows in and an outlet through which the air is discharged. The freshness maintenance device may also include an electromagnetic wave module, installed at the inlet of the main body, for ionizing food freshness reducing materials included in the air by emitting electromagnetic waves to the air. The device may further include a collection module, installed at the outlet of the main body, for collecting the ionized food freshness reducing materials from the air.

### CROSS REFERENCE TO RELATED APPLICATIONS

This is a continuation application of International Patent Application No. PCT/KR2021/016987, filed on Nov. 18, 2021, which claims priority to Korean patent application No. 10-2021-0159029 filed on Nov. 18, 2021, contents of both of which are incorporated herein by reference in their entireties.

### BACKGROUND

### Technical Field

The present disclosure relates to a food freshness maintenance system and a freshness maintenance and transfer device.

### Description of Related Technology

In general, fungi such as mold, and viruses, bacteria, etc. may remain on the surface of food such as vegetables and fruits and they may reduce the freshness of food by causing food that is in storage to go rotten and to be contaminated. In particular, mold has a problem in that mold may cause diseases or abnormal physiological actions of humans or livestock by additionally producing mold toxins.

### SUMMARY

Embodiments of the present disclosure have been made under the background described above to provide a food freshness maintenance system and a freshness maintenance and transfer device that can maintain freshness of food through general freshness management against materials that reduce freshness such as fungi such as mold, ethylene gas, viruses, and bacteria existing in the air in a storage space.

In accordance with an embodiment of the present disclosure, there may be provided a food freshness maintenance system including: a chamber configured to accommodate food; and a freshness maintenance device installed in the chamber, wherein the freshness maintenance device includes: a main body including an inlet through which air flows in and an outlet through which the air is discharged; an electromagnetic wave module, installed at the inlet of the main body, for ionizing food freshness reducing materials included in the air by emitting electromagnetic waves to the air; and a collection module, installed at the outlet of the main body, for collecting the ionized food freshness reducing materials from the air. Further, the freshness maintenance device may further include an adsorption module having a porous material adsorbing the ionized food freshness reducing materials from the air that has passed through the collection module.

Further, the freshness maintenance device may further include a blowing module providing a blowing force to move air in the chamber into the main body.

Further, the freshness maintenance device may further include a filter module installed between the blowing module and the inlet of the main body, for filtering out foreign substances of a preset particle size or more from the air flowing into the main body.

Further, the freshness maintenance device may further include a voltage generation module for applying a voltage to the electromagnetic wave module, and the electromagnetic wave module may include a plurality of electromagnetic wave tubes spaced apart from each other at both sides in the main body and emitting electromagnetic waves having a wavelength in the range of 0.1 nm to 10 nm toward the air by receiving the voltage from the voltage generation module.

Further, the collection module may include: a plurality of first collection plates spaced apart from each other in one direction in an internal space of the main body; a plurality of second collection plates disposed between the plurality of first collection plates; a first electrode electrically connected with the first collection plate; and a second electrode connected with the second collection plate to provide a voltage, which is different from a voltage of the first electrode, to the second collection plate such that a voltage difference is generated between the first collection plate and the second collection plate

Further, the collection module may include: a plurality of first collection plates spaced apart from each other in one direction in an internal space of the main body; a second collection plate disposed between the plurality of first collection plates and being a grounding electrode made of a porous material; and a first electrode electrically connected with the plurality of first collection plates to provide a negative voltage or a positive voltage to the plurality of first collection plates.

Further, the food freshness reducing materials may include fungi including mold, viruses, bacteria, and ripening acceleration gas

Further, the ripening acceleration gas may include ethylene gas

Further, the electromagnetic wave module may emit electromagnetic waves to the air such that ozone is not produced, and the collection module may collect ionized food freshness reducing materials from the air without production of ozone.

In accordance with an embodiment of the present disclosure, there may be provided a freshness maintenance and transfer device including: the food freshness maintenance system described above; and a transfer vehicle configured to be movable with the food freshness maintenance system mounted thereon.

According to embodiments of the present disclosure, there are the effects that it is possible to ionize food freshness reducing materials existing in the air in a food storage space and effectively collect the ionized food freshness reducing materials through a collection module and an adsorption module, and accordingly, it is possible to effectively increase the function of keeping food.

Further, according to embodiments of the present disclosure, there is the effect that it is possible to improve the efficiency of adsorbing ethylene gas by ionizing ethylene gas produced from food.

Further, according to embodiments of the present disclosure, there are the effects that it is possible to directly inactivate viruses in air, kill bacteria and fungi, and ionize viruses, bacteria, and fungi in air using electromagnetic waves emitted through an electromagnetic wave tube, and it is possible to collect foreign substances in air using an electrostatic force, and accordingly, it is possible to achieve general freshness management against ethylene gas, viruses, bacteria, bacilli, and fungi.

Further, according to embodiments of the present disclosure, since electromagnetic waves of an electromagnetic wave tube are used to manage freshness of food, it is possible to prevent production of ozone, it is possible to solve the problem of harmfulness of ozone in comparison to sterilizer-purifiers that use ozone in the related art, and it is not required to install an ozone remover for preventing ozone from being discharged outside.

### BRIEF DESCRIPTION OF THE DRAWINGS

FIG. 1 is a configuration view showing a freshness maintenance system according to an embodiment of the present disclosure.

FIG. 2 is an assembled perspective view showing a freshness maintenance device of the freshness maintenance system according to the embodiment of the present disclosure.

FIG. 3 is an exploded perspective view showing the freshness maintenance device of the freshness maintenance system according to the embodiment of the present disclosure.

FIG. 4 is an enlarged perspective view enlarging and showing a collection module of the freshness maintenance device according to the embodiment of the present disclosure.

FIG. 5 is an enlarged side view enlarging and showing the collection module of the freshness maintenance device according to the embodiment of the present disclosure.

FIG. 6 is a configuration view showing a freshness maintenance and transfer device according to the embodiment of the present disclosure.

FIG. 7 is a graph showing the number of dust particles over time in the freshness maintenance system according to the embodiment of the present disclosure.

FIG. 8A and FIG. 8B are pictures comparing the concentration of ethylene gas in a space to which the freshness maintenance device according to the embodiment of the present disclosure has been applied and a space to which the freshness maintenance device has not been applied.

FIG. 9A and FIG. 9B are pictures for observing the influence on food by the freshness maintenance device according to the embodiment of the present disclosure in terms of whether mold transfers to other fruits.

### DETAILED DESCRIPTION

When food such as vegetables or fruits is kept, ethylene gas is produced from vegetables or fruits. Ethylene gas is a kind of gas that is produced while vegetables or fruits ripen. Since ethylene gas acts as an aging acceleration hormone that accelerates aging of vegetables or fruits, ethylene gas may reduce freshness and keeping quality of food.

Accordingly, there is a need for a technology that can remove fungi such as mold, which reduce freshness while floating in the air in the space in which food is kept, and can effectively remove ethylene gas produced from food, viruses, bacteria, bacilli, and fungi, etc. that remain in food.

Hereinafter, specific embodiments for implementing a spirit of the present disclosure will be described in detail with reference to the drawings.

In describing the present disclosure, detailed descriptions of known configurations or functions may be omitted to clarify the present disclosure.

When an element is referred to as being ‘connected’ to, ‘supported’ by, ‘accessed’ to, ‘supplied’ to, ‘transferred’ to, or ‘contacted’ with another element, it should be understood that the element may be directly connected to, supported by, accessed to, supplied to, transferred to, or contacted with another element, but that other elements may exist in the middle.

The terms used in the present disclosure are only used for describing specific embodiments, and are not intended to limit the present disclosure. Singular expressions include plural expressions unless the context clearly indicates otherwise.

Further, in the present disclosure, it is to be noted that expressions, such as the upper side and the lower side, are described based on the illustration of drawings, but may be modified if directions of corresponding objects are changed. For the same reasons, some components are exaggerated, omitted, or schematically illustrated in the accompanying drawings, and the size of each component does not fully reflect the actual size.

Terms including ordinal numbers, such as first and second, may be used for describing various elements, but the corresponding elements are not limited by these terms. These terms are only used for the purpose of distinguishing one element from another element.

In the present specification, it is to be understood that the terms such as “including” are intended to indicate the existence of the certain features, areas, integers, steps, actions, elements, combinations, and/or groups thereof disclosed in the specification, and are not intended to preclude the possibility that one or more other certain features, areas, integers, steps, actions, elements, combinations, and/or groups thereof may exist or may be added.

Hereafter, the detailed configuration of a food freshness maintenance system according to the present disclosure is described.

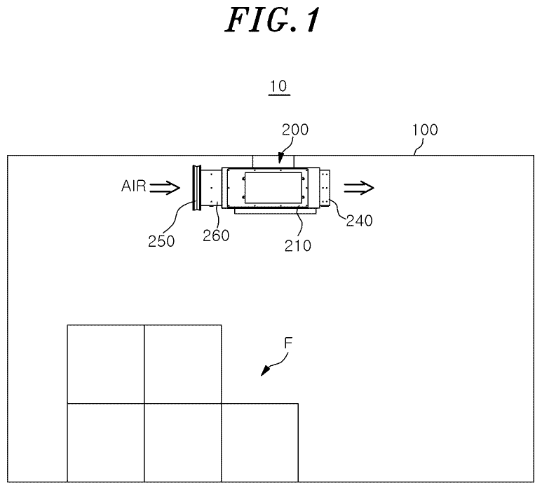

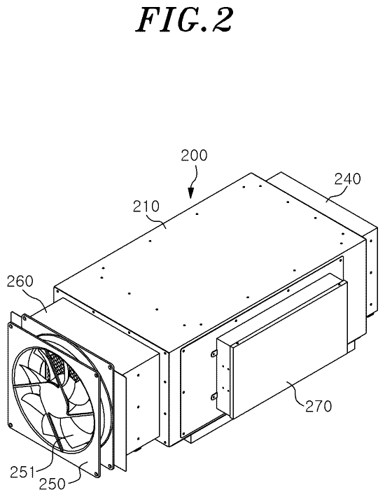

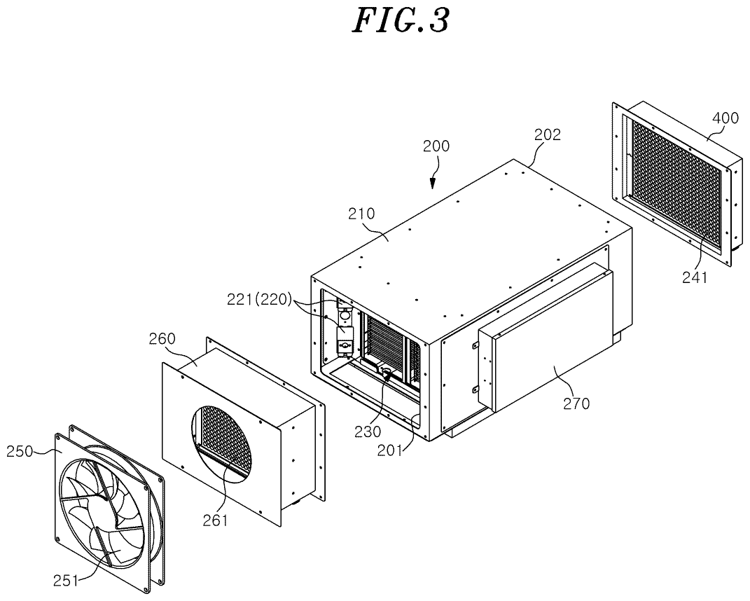

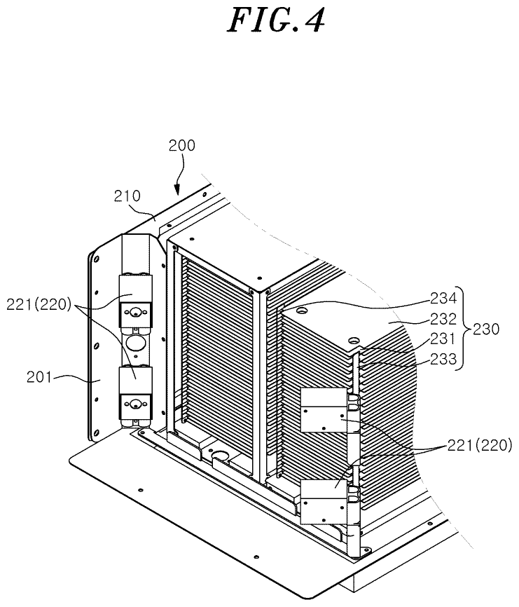

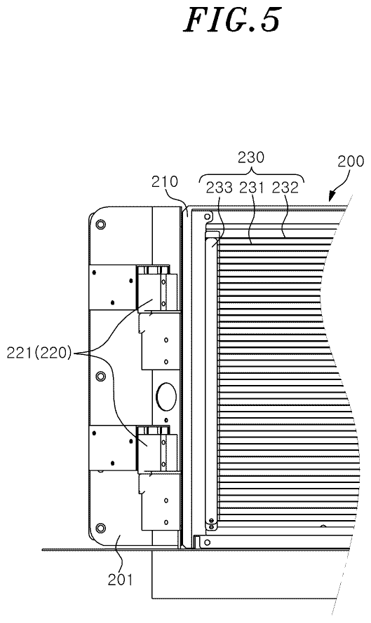

FIG. 1 is a configuration view showing a freshness maintenance system according to an embodiment of the present disclosure, FIG. 2 is an assembled perspective view showing a freshness maintenance device of the freshness maintenance system according to the embodiment of the present disclosure, FIG. 3 is an exploded perspective view showing the freshness maintenance device of the freshness maintenance system according to the embodiment of the present disclosure, FIG. 4 is an enlarged perspective view enlarging and showing a collection module of the freshness maintenance device according to the embodiment of the present disclosure, and FIG. 5 is an enlarged side view enlarging and showing the collection module of the freshness maintenance device according to the embodiment of the present disclosure.

Referring to FIGS. 1 to 5, a food freshness maintenance system 10 according to the embodiment of the present disclosure can maintain freshness of food F through general freshness management for the food F. In order to implement this, the freshness maintenance system 10 may include a chamber 100 in which food F can be accommodated and a freshness maintenance device 200 installed in the chamber 100.

In detail, the chamber 100 can provide a space having a predetermined capacity. Food F such as fruits and vegetables can be accommodated in the chamber. For example, the chamber 100 may be a container box type that can be installed on a transfer vehicle (a box truck, a truck, etc.), in a warehouse, or the like.

The freshness maintenance device 200 for maintaining freshness of food F can be installed in the chamber 100. In the embodiment, the freshness maintenance device 200 is installed at the upper portion (on the ceiling) in the chamber 100 but is not limited thereto, and the freshness maintenance device 200 may be installed at various positions in the chamber 100 to effectively maintain freshness of food F. For example, the freshness maintenance device 200 may be installed on the floor, a side, or the like of the chamber 100.

The freshness maintenance device 200 may include a main body 210, an electromagnetic wave module 220, a collection module 230, an adsorption module 240, a blowing module 250, a filter module 260, and a voltage generation module 270.

The main body 210 may be provided in a box shape. An inlet 201 for suctioning air may be disposed at a front end of the main body 210 and an outlet 202 for discharging air may be disposed at a rear end of the main body 210. The main body 210 can provide an internal space for maintaining freshness by sterilizing and purifying air.

The electromagnetic wave module 220 and the collection module 230 can be installed in the internal space of the main body 210 and the voltage generation module 270 can be mounted on a side of the main body 210. Further, the blowing module 250 and the filter module 260 can be installed at the front end of the main body 210 and the adsorption module 240 can be mounted at the rear end of the main body 210. The blowing module 250, the filter module 260, the electromagnetic wave module 220, the collection module 230, and the adsorption module 240 can be sequentially disposed in a line from the front to the rear of the main body 210.

Accordingly, when the air in the chamber 100 is circulated through the freshness maintenance device 200, food freshness reducing materials such as fungi such as mold, ripening acceleration gas, viruses, and bacteria can be removed while the air sequentially flows through the blowing module 250, the filter module 260, the electromagnetic wave module 220, the collection module 230, and the adsorption module 240. Accordingly, freshness of food F in the chamber 100 can be improved.

In this case, mold that reduces freshness of food may include Mucor genus, Rhizopus genus, Penicillium genus, Neurospora genus, Aspergillus genus, and Fusarium genus.

Further, in this case, the ripening acceleration gas may include ethylene gas. Ethylene gas is exemplified as ripening acceleration gas in the embodiment, but the ripening acceleration gas is not limited thereto and may include various kinds of hormone gas that can quickly accelerate ripening of food or can reduce freshness of food. For example, the ripening acceleration gas may be methyl jasmonate.

The electromagnetic wave module 220 may be installed at the inlet 201 of the main body 210. For example, the electromagnetic wave module 220 may be disposed at the upstream side further than the collection module 230 and may emit electromagnetic waves before air is collected in the collection module 230. The electromagnetic wave module 220 can emit electromagnetic waves by receiving a high voltage (e.g., 0.1 Kv˜15 Kv) from the voltage generation module 270.

The electromagnetic wave module 220 can emit electromagnetic waves to air such that ozone is not produced or is produced only in a very small amount under a predetermined range. For example, when the electromagnetic wave module 220 uses a voltage of 0.1 kV to 15 kV required for photo ionization, plasma is not produced in the corresponding energy range, and ozone is not produced because the wavelength is short in comparison to the range of UV-C. The electric field between a first collection plate 231 and a second collection plate 232 can be maintained at 0.1 kV/mm to 1 kV/mm.

This is because when high-energy electromagnetic waves (x-rays) are generated by accelerating electrons to a high voltage, ozone may be produced by production of plasma, and even when the electric field between the first collection plate 231 and the second collection plate 232 becomes larger than 1 kV/mm, ozone may be produced. In the embodiment, the electric field between the first collection plate 231 and the second collection plate 232 may be 0.1 kV/mm to 50 kV/mm.

Meanwhile, as another example, the electromagnetic wave module 220 can emit electromagnetic waves to air such that ozone is produced only by a very small amount under a predetermined range. For example, an electromagnetic wave module 220 may emit electromagnetic waves to air such that ozone is produced under 50 ppb. When ozone is produced under 50 ppb, the electric field between the first collection plate 231 and the second collection plate 232 may be 1 kV/mm to 5 kV/mm.

Accordingly, fungi such as mold, ethylene gas, viruses, and bacteria in the air moving in the internal space of the main body 210 can be ionized without production of ozone. Further, viruses in air can be directly inactivated by electromagnetic waves, and fungi such as mold, and bacteria in air can be directly killed by electromagnetic waves.

For example, when molecules are exposed to light corresponding to the range of electromagnetic waves, particularly, soft x-rays, photoionization of losing one electron may be generated. In the embodiment, ethylene gas in the air moving in the internal space of the main body 210 can be ionized, as in the following chemical equation 1, by electromagnetic waves from the electromagnetic wave module 220.

$${C}_{2} {H}_{4} + hv \to {C}_{2} {H}_{4} {n}^{+} + {n}^{-} \qquad ( CHEMICAL EQUATION 1 )$$

Chemical equation 1 can be equally applied to fungi such as mold, viruses, and bacteria as well. Since all materials that are different kinds but are molecules or particles that can emit free electrons by light, such ionization may occur when they are exposed to electromagnetic waves such as soft x-rays.

The electromagnetic wave module 220 may include an electromagnetic wave tube 221. The electromagnetic wave tube 221 receives a high voltage from the voltage generation module 270, thereby being able to emit electromagnetic waves having a wavelength in the range of 0.1˜10 nm. In this case, electromagnetic waves may be provided in the type of ultraviolet rays, x-rays, and Extreme UltraViolet (EUV) through adjustment of an electromagnetic wavelength.

The electromagnetic wave tube 221 can emit electromagnetic waves, which are generated when electrons discharged from an emitter (e.g., a Carbon Nanotube (CNT)-based emitter) hit a target at a high speed, to air. An emitter can adjust an electromagnetic wavelength such that the intensity of electromagnetic waves changes by changing (adjusting) the tube voltage of the electromagnetic waves, and the tube voltage may be understood as the maximum voltage given between the anode and the cathode of the electromagnetic wave tube 221. When the tube voltage of electromagnetic waves is adjusted, the wavelength, the radiation quality, the radiation dose, etc. of the electromagnetic waves that are generated from the electromagnetic wave tube 221 are changed, so the penetration ability, etc. of the electromagnetic waves may change.

A plurality of electromagnetic wave tubes 221 may be provided in the main body 210. The plurality of electromagnetic wave tubes 221 is spaced apart from each other at both sides in the main body 210, whereby it is possible to emit electromagnetic waves to channels through which air moves.

For example, a pair of electromagnetic wave tubes 221 may be spaced apart from each other in the up-down direction of the main body 210 at the left side in the main body 210 and another pair of electromagnetic wave tubes 221 may be spaced apart from each other in the up-down direction of the main body 210 at the right side in the main body 210. A pair of electromagnetic wave tubes 221 may be positioned at the same height at both sides in the main body 210 and may emit electromagnetic waves to air moving in the internal space of the main body 210. In particular, a pair of electromagnetic wave tubes 221 disposed at both sides in the main body 210 may be disposed to be inclined toward the front of the main body 210 in the front-rear direction of the main body 210.

In the embodiment, a pair of electromagnetic wave tubes 221 is spaced apart from each other at each of both sides in the main body 210, but the number/position of the electromagnetic wave tubes 221 is not limited thereto and may be variously changed.

The collection module 230 may be installed at the outlet 202 of the main body 210. The collection module 230 can collect ionized ethylene gas from air without production of ozone. Further, the collection module 230 can collect ionized viruses, bacteria, mold, foreign substances, etc. by providing electrostatic force thereto.

The collection module 230 may include a first collection plate 231, a second collection plate 232, a first electrode 233, and a second electrode 234. The first collection plate 231 and the second collection plate 232 may be collection plates that can be charged such that a voltage difference is generated between the first collection plate 231 and the second collection plate 232. The first electrode 233 and the second electrode 234 may be electrodes that provide different voltages such that a voltage difference is generated between the first collection plate 231 and the second collection plate 232. This is because when a voltage difference exists between the first collection plate 231 and the second collection plate 232, the force that attracts contaminants in air depends on the difference of electromotive force per distance due to the electrostatic force between the first collection plate 231 and the second collection plate 232.

A plurality of first collection plates 231 spaced apart from each other in one direction, in more detail, in the up-down direction of the main body 210 may be provided. The plurality of first collection plates 231 may be spaced a predetermined distance apart from each other. For example, when a negative voltage is applied to the first collection plate 231 from the first electrode 233, the first collection plate 231 may function as a negative plate.

The second collection plate 232 may be disposed between a plurality of first collection plates 231. For example, when a positive voltage is applied to the second collection plate 232 from the second electrode 234, the second collection plate 232 may function as a positive plate by receiving a positive voltage from the second electrode 234.

The first electrode 233 may be disposed to extend in the up-down direction in one side of the main body 210. The first electrode 233 may be electrically connected with one side edges of a plurality of first collection plates 231. The first electrode 233 can provide a voltage, which is different from that of the second electrode 234, to the first collection plate 231 such that a voltage difference is generated between the first collection plate 231 and the second collection plate 232. For example, the first electrode 233 may provide a negative voltage to a plurality of first collection plates 231.

The second electrode 234 may be disposed to extend in the up-down direction on a side of the main body 210. The second electrode 234 may be electrically connected with other side edges of a plurality of second collection plates 232. The second electrode 234 can provide a voltage, which is different from that of the first electrode 233, to the second collection plate 232 such that a voltage difference is generated between the first collection plate 231 and the second collection plate 232. For example, the second electrode 234 may provide a positive voltage to a plurality of second collection plates 232.

The first electrode 233 provides a negative voltage to the first collection plate 231 and the second electrode 234 provides a positive voltage to the second collection plate 232 in the embodiment, but, in order to generate a voltage difference between the first collection plate 231 and the second collection plate 232, the present disclosure is not limited thereto and the first electrode 233 and the second electrode 234 may provide various voltages to the first collection plate 231 and the second collection plate 232. For example, the first electrode 233 may provide a negative voltage to the first collection plate 231 and the second electrode 234 may be used as a grounding electrode, or the first electrode 233 may provide a positive voltage to the first collection plate 231 and the second electrode 234 may be used as a grounding electrode.

According to a modified example of the present disclosure, the collection module 230 may include a first collection plate 231, a second collection plate 232 that is a grounding electrode providing a grounding function, and a first electrode 233 providing a negative voltage or a positive voltage to the first collection plate 231.

In this case, the first collection plate 231 may perform the function of a negative plate or a positive plate by receiving a negative voltage or a positive voltage from the first electrode 233 and the second collection plate 232 may be a grounding electrode including a porous material for adsorbing ripening acceleration gas. In the modified example, since a voltage difference may be generated between the first collection plate 231 that performs the function of a negative plate or a positive plate and the second collection plate 232 that provides a grounding function, the second electrode 234 described in the above embodiment may not be needed.

The adsorption module 240 may be installed at the rear end of the main body 210. The adsorption module 240 may include an adsorption filter 241 for adsorbing ionized ripening acceleration gas. Ionized ethylene gas and contaminants can be adsorbed to the adsorption filter 241.

The adsorption filter 241 may be made of a porous material. The porous material may include any one or more of activated carbon, zirconium, a Metal-organic Framework (MOF), a covalent organic framework (COF), zeolitic imidazolate frameworks (ZIF), or covalent organic frameworks (COF).

The blowing module 250 may be installed at the front end of the main body 210. The blowing module 250 may include a blowing fan 251 that blows the air in the chamber 100 to the internal space of the main body 210. The air in the chamber 100 that keeps food F can be forcibly circulated to the freshness maintenance device 200 by the blowing operation of the blowing module 250.

The filter module 260 may be installed between the blowing module 250 and the inlet 201 of the main body 210. The filter module 260 may include a filter 261 that filters out foreign substances over a preset particle. Accordingly, the filter module 260 can remove foreign substances over a preset particle from the air flowing into the main body 210.

As described above, since the filter module 260 removes large-size foreign substances in advance from the air before flowing into the main body 210, it is possible to improve the collection efficient of the collection module 230 and the adsorption efficient of the adsorption module 240.

The voltage generation module 270 may be installed on a side of the main body 210. The voltage generation module 270 can apply a high voltage to the electromagnetic wave module 220. For example, the voltage generation module 270 can generate a high voltage of 0.5 kV˜20 kV. Further, the voltage generation module 270 may also provide a negative voltage and a positive voltage to the first electrode 233 and the second electrode 234.

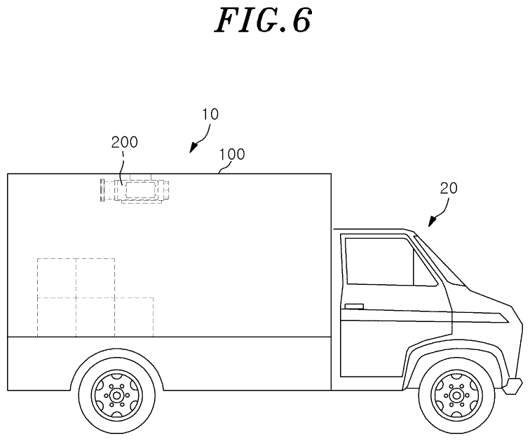

FIG. 6 is a configuration view showing a freshness maintenance and transfer device according to an embodiment of the present disclosure.

Referring to FIG. 6, the freshness maintenance and transfer device of the prevent disclosure may include a freshness maintenance system 10 and a transfer vehicle 20. In this case, the freshness maintenance system 10 is the same in configuration as the freshness maintenance system 10 described above, so detailed description thereof is omitted.

The transfer vehicle 20 may be a means of transportation that can be moved. The freshness maintenance system 10 may be mounted on the transfer vehicle 20. In the embodiment, the transfer vehicle 20 may be a box truck or a truck on which the freshness maintenance system 10 can be mounted, but, other than a box truck or a truck, the transfer vehicle 20 may be various kinds of vehicles that can be moved with the freshness maintenance system 10 mounted thereon.

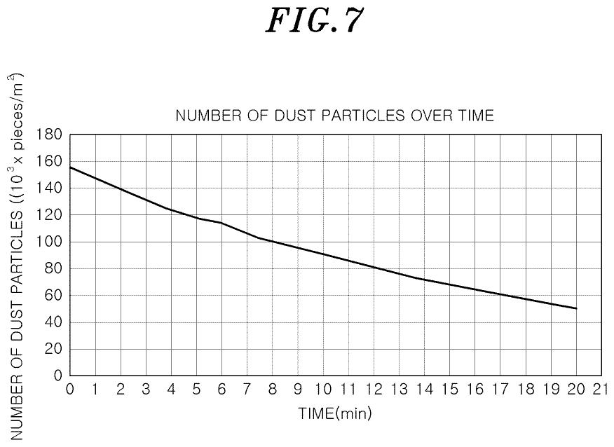

FIG. 7 is a graph showing the number of dust particles over time in the freshness maintenance system according to the embodiment of the present disclosure.

Referring to FIG. 7, the number of dust particles over time in the chamber 100 was measured through the freshness maintenance device 200 according to the present disclosure.

The test results for respective test items are as in the following Table 1 (test conditions for respective test items: temperature 21±1(° C.), humidity 45±5(%) R.H.).

TABLE 1

| Item | Purification ability (m³/min) | Area of Application (m²) |
|---|---|---|
| Test results | 1.6 | 12.6 |

According to the test results for respective test items, it could be seen that dust in the chamber rapidly decreases over time.

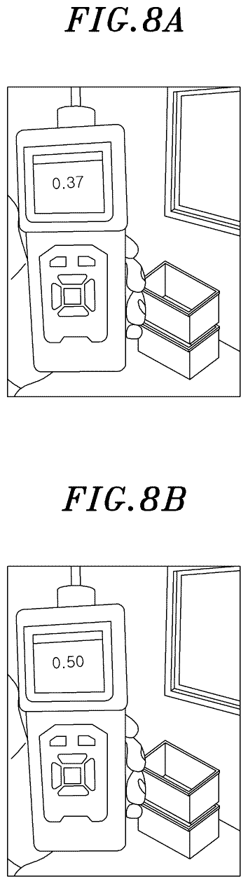

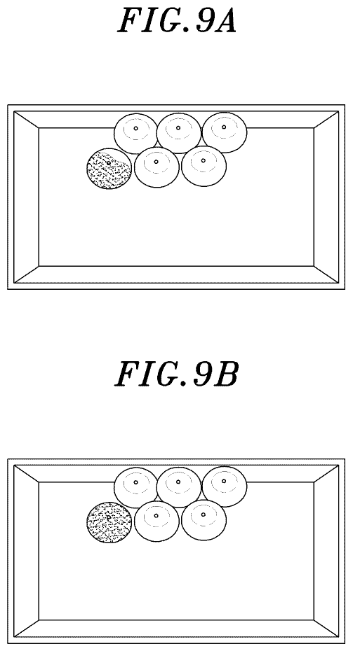

FIG. 8A and FIG. 8B are views comparing the concentration of ethylene gas in a space to which the freshness maintenance device according to the embodiment of the present disclosure has been applied and a space to which the freshness maintenance device has not been applied and FIG. 9A and FIG. 9B are pictures for observing the influence on food by the freshness maintenance device according to the embodiment of the present disclosure in terms of whether mold transfers to other fruits.

Referring to FIG. 8A and FIG. 8B, food (Hwang Geum Hyang (Beni Madonna)) was kept for the same period under the same conditions (temperature, pressure, humidity, etc.) in a space to which the freshness maintenance device 200 was applied (see FIG. 8A) and a space to which the freshness maintenance device 200 was not applied (see FIG. 8B).

As the result of comparing the space to which the freshness maintenance device 200 according to the embodiment of the present disclosure was applied (see FIG. 8A) and the case to which the freshness maintenance device 200 according to the embodiment of the present disclosure was not applied (see FIG. 8B), it could be seen that there is a difference in concentration of ethylene gas, depending on whether the freshness maintenance device 200 according to the present disclosure is installed or not.

For example, the concentration of ethylene gas detected in the space to which the freshness maintenance device 200 was applied (see FIG. 8A) was 0.37 PPM but the concentration of ethylene gas detected in the space to which the freshness maintenance device 200 was not applied (see FIG. 8B) was 0.50 PPM.

Referring to FIG. 8A to FIG. 9B, it could be seen that even though one moldy Hwang Geum Hyang was placed among a plurality of Hwang Geum Hyangs, the mold on one Hwang Geum Hyang did not transfer to other Hwang Geum Hyangs in the space in which the freshness maintenance device 200 according to the present disclosure was installed.

For example, after a predetermined time (seven days) passed with one moldy Hwang Geum Hyang and five fresh Hwang Geum Hyangs kept in a space in which the freshness maintenance device 200 was installed (see FIG. 9A), whether the mold on one Hwang Geum Hyang transferred to other Hwang Geum Hyangs was observed.

As the result, as shown in FIG. 9B, it could be seen that the mold on the moldy one moldy Hwang Geum Hyang became worse but the other five Hwang Geum Hyangs were kept fresh without mold transferred thereto.

As described above, the present disclosure has the advantages in that it is possible to effectively collect ionized food freshness reducing materials produced from food through a collection module and an adsorption module by ionizing the food freshness reducing materials, it is possible to improve the efficiency of adsorbing ethylene gas produced from food by ionizing the ethylene gas, it is possible to achieve general freshness management against ethylene gas, viruses, bacteria, bacilli, and fungi, it is possible to be free from the problem of harmfulness of ozone because the present disclosure does not produce ozone by using electromagnetic waves from an electromagnetic tube, etc.

The examples of the present disclosure have been described above as specific embodiments, but these are only examples, and the present disclosure is not limited thereto, and should be construed as having the widest scope according to the technical spirit disclosed in the present specification. A person skilled in the art may combine/substitute the disclosed embodiments to implement a pattern of a shape that is not disclosed, but it also does not depart from the scope of the present disclosure. In addition, those skilled in the art can easily change or modify the disclosed embodiments based on the present specification, and it is clear that such changes or modifications also belong to the scope of the present disclosure.

What is claimed is:

1. A food freshness maintenance system comprising:
a chamber configured to accommodate food; and
a freshness maintenance device installed in the chamber,
wherein the freshness maintenance device includes:
a main body including an inlet through which air flows in and an outlet through which the air is discharged;
an electromagnetic wave module, installed at the inlet of the main body, configured to ionize food freshness reducing materials included in the air by emitting electromagnetic waves to the air; and
a collection module, installed at the outlet of the main body, configured to collect the ionized food freshness reducing materials from the air.

2. The food freshness maintenance system of claim 1, wherein the freshness maintenance device further includes an adsorption module having a porous material configured to adsorb the ionized food freshness reducing materials from the air that has passed through the collection module.

3. The food freshness maintenance system of claim 1, wherein the freshness maintenance device further includes a blowing module configured to provide a blowing force to move air in the chamber into the main body.

4. The food freshness maintenance system of claim 3, wherein the freshness maintenance device further includes a filter module installed between the blowing module and the inlet of the main body, configured to filter out foreign substances of a preset particle size or more from the air flowing into the main body.

5. The food freshness maintenance system of claim 1, wherein the freshness maintenance device further includes a voltage generation module configured to apply a voltage to the electromagnetic wave module, and
wherein the electromagnetic wave module includes a plurality of electromagnetic wave tubes spaced apart from each other at both sides in the main body and configured to emit electromagnetic waves having a wavelength in the range of 0.1 nm to 10 nm toward the air by receiving the voltage from the voltage generation module.

6. The food freshness maintenance system of claim 1, wherein the collection module includes:
a plurality of first collection plates spaced apart from each other in one direction in an internal space of the main body;
a plurality of second collection plates disposed between the plurality of first collection plates;
a first electrode electrically connected with the first collection plate; and
a second electrode connected with the second collection plate to provide a voltage, which is different from a voltage of the first electrode, to the second collection plate such that a voltage difference is generated between the first collection plate and the second collection plate.

7. The food freshness maintenance system of claim 1, wherein the collection module includes:
a plurality of first collection plates spaced apart from each other in one direction in an internal space of the main body;
a second collection plate disposed between the plurality of first collection plates and being a grounding electrode made of a porous material; and
a first electrode electrically connected with the plurality of first collection plates to provide a negative voltage or a positive voltage to the plurality of first collection plates.

8. The food freshness maintenance system of claim 1, wherein the food freshness reducing materials include fungi including mold, viruses, bacteria, and ripening acceleration gas.

9. The food freshness maintenance system of claim 8, wherein the ripening acceleration gas includes ethylene gas.

10. The food freshness maintenance system of claim 1, wherein the electromagnetic wave module is configured to emit electromagnetic waves to the air such that ozone is not produced, and the collection module collects ionized food freshness reducing materials from the air without production of ozone.

11. A freshness maintenance and transfer device comprising:
the food freshness maintenance system of claim 1; and
a transfer vehicle configured to be movable with the food freshness maintenance system mounted thereon.
